(19) United States
(12) Patent Application Publication
(10) Pub. No.: US 2026/0233046 A1
(43) Pub. Date: Aug. 13, 2026

(54) AZEOTROPE OR AZEOTROPE-LIKE COMPOSITIONS OF TRIFLUOROIODOMETHANE (CF3I) AND WATER

(71) Applicant: Honeywell International Inc., Charlotte, NC (US)

(72) Inventors: Haluk Kopkalli, Staten Island, NY (US); Jennifer W. McClaine, Branchburg, NJ (US); Tao Wang, Shanghai (CN); Richard Wilcox, West Caldwell, NJ (US); Yuon Chiu, Denville, NJ (US); Hang T. Pham, Amherst, NY (US)

(21) Appl. No.: 19/177,805

(22) Filed: Apr. 14, 2025

Related U.S. Application Data
(63) Continuation of application No. 18/400,106, filed on Dec. 29, 2023, now abandoned.
Application No. 18/400,106 is a continuation of application No. 17/466,694, filed on Sep. 3, 2021, now abandoned.
(60) Provisional application No. 63/077,358, filed on Sep. 11, 2020.

(51) Int. Cl. A62D 1/00; B01D 3/14; B01D 3/36; B01D 11/04; C08J 9/12; C08J 9/14; C09K 3/30; C09K 5/04

(52) U.S. Cl. CPC A62D 1/0057; B01D 3/143; B01D 3/36; B01D 11/04; C08J 9/125; C08J 9/127; C08J 9/147; C09K 3/30; C09K 5/044; C09K 5/048; C08J 2203/10; C08J 2203/144; C08J 2203/182; C09K 2205/128; C09K 2205/24; C09K 2205/32

(57) ABSTRACT

Heterogeneous azeotrope or azeotrope-like compositions comprising trifluoroiodomethane (CF₃I) and water which may include from about 47.7 wt. % to about 99.0 wt. % trifluoroiodomethane (CF₃I) and from about 1.0 wt. % to about 52.3 wt. % water and having a boiling point between about 18.0° C. and about 19.0° C. at a pressure of between about 58.0 psia and about 60.0 psia. The azeotrope or azeotrope-like compositions may be used to separate impurities from trifluoroiodomethane (CF₃I).

### CROSS-REFERENCE TO RELATED APPLICATIONS

This application is a continuation application of U.S. patent application Ser. No. 18/400,106, filed Dec. 29, 2023, which is a continuation application of U.S. patent application Ser. No. 17/466,694, filed Sep. 3, 2021, which claims priority to U.S. Provisional Application No. 63/077,358, filed Sep. 11, 2020, each of which are herein incorporated by reference in their entireties.

### FIELD

The present disclosure pertains to azeotrope or azeotrope-like compositions and, in particular, azeotrope or azeotrope-like compositions comprising effective amounts of trifluoroiodomethane (CF₃I) and water.

### BACKGROUND

Fluorocarbon based fluids have found widespread use in industry in a number of applications, including as refrigerants, aerosol propellants, blowing agents, heat transfer media, gaseous dielectrics, and fire suppression.

The industry is continually seeking new fluorocarbon-based mixtures that offer alternatives, and are considered environmentally safer substitutes for CFCs, HCFCs and HFCs in use today. Of particular interest are mixtures containing hydrofluorocarbons, fluoroolefins, iodide containing compounds and other fluorinated compounds, which have low ozone depletion potentials and low global warming potentials. Such mixtures are the subject of this disclosure.

Although iodide containing compounds are of great potential interest, the purification of iodide containing compounds such as trifluoroiodomethane (CF₃I) has presented challenges, and techniques for the removal of impurities from trifluoroiodomethane (CF₃I) are in demand.

### SUMMARY

The present disclosure provides heterogeneous azeotrope or azeotrope-like compositions of trifluoroiodomethane (CF₃I) and water.

It is well-recognized in the art that it is not possible to predict the formation of azeotropes, and the present inventors have discovered unexpectedly that trifluoroiodomethane (CF₃I) and water form azeotrope or azeotrope-like compositions and, in particular, form heterogeneous azeotrope or azeotrope-like compositions.

The present disclosure provides a composition comprising an azeotrope or azeotrope-like composition comprising, consisting essentially of, or consisting of effective amounts of trifluoroiodomethane (CF₃I) and water.

The azeotrope or azeotrope-like composition may comprise from about 47.7 wt. % to about 99.0 wt. % trifluoroiodomethane (CF₃I) and from about 1.0 wt. % to about 52.3 wt. % water, from about 60.4 wt. % to about 95.0 wt. % trifluoroiodomethane (CF₃I) and from about 5.0 wt. % to about 39.6 wt. % water, from about 70.2 wt. % to about 90.0 wt. % trifluoroiodomethane (CF₃I) and from about 10.0 wt. % to about 29.8 wt. % water, or the azeotrope or azeotrope-like composition may consist essentially of about 77.0 wt. % trifluoroiodomethane (CF₃I) and about 23.0 wt. % water. The azeotrope or azeotrope-like composition may consist essentially of trifluoroiodomethane (CF₃I) and water in the above amounts or consist of trifluoroiodomethane (CF₃I) and water in the above amounts.

The azeotrope of azeotrope-like composition has a boiling point between about 18.0° C. and about 19.0° C. at a pressure of between about 58.0 psia and about 60.0 psia.

In a further form thereof, the present disclosure provides a method of forming an azeotrope or azeotrope-like composition comprising the step of combining trifluoroiodomethane (CF₃I) and water to form an azeotrope or azeotrope-like composition comprising, consisting essentially of, or consisting of trifluoroiodomethane (CF₃I) and water. The azeotrope of azeotrope-like composition may have a boiling point between about 18.0° C. and about 19.0° C. at a pressure of between about 58.0 psia and about 60.0 psia.

The present disclosure further provides a method of separating impurities from a composition which includes trifluoroiodomethane (CF₃I), water, and at least one impurity, comprising the steps of modifying the relative amounts of trifluoroiodomethane (CF₃I) and water and subjecting the composition to conditions effective to form an azeotrope or azeotrope-like composition consisting essentially of, or consisting of, effective amounts of trifluoroiodomethane (CF₃I) and water; and separating the azeotrope or azeotrope-like composition from the at least one impurity, wherein the separation step may comprise at least one of phase separation, distillation, and fractionation.

The present disclosure further provides a method of separating impurities from a composition which includes trifluoroiodomethane (CF₃I) and at least one impurity, comprising the steps of adding an effective amount of water to the composition; modifying the relative amounts of trifluoroiodomethane (CF₃I) and water and subjecting the composition to conditions effective to form an azeotrope or azeotrope-like composition consisting essentially of, or consisting of, effective amounts of trifluoroiodomethane (CF₃I) and water; and separating the azeotrope or azeotrope-like composition from the at least one impurity, wherein the separation step may comprise at least one of phase separation, distillation, and fractionation.

In the foregoing methods, the step of modifying the relative amounts of trifluoroiodomethane (CF₃I) and water may involve adding trifluoroiodomethane (CF₃I) to the composition, adding water to the composition, or adding both trifluoroiodomethane (CF₃I) and water to the composition.

Following the separation, the composition may be altered in its characteristics such that the water may be removed from the composition and the trifluoroiodomethane (CF₃I) may be further purified. Suitable methods to purify the trifluoroiodomethane (CF₃I) may include distillation, liquid-liquid extraction, or exposure to a drying agent.

### DETAILED DESCRIPTION

It has been found that trifluoroiodomethane (CF₃I) forms heterogeneous azeotropic and azeotrope-like compositions or mixtures with water, and the present disclosure provides heterogeneous azeotropic or azeotrope-like compositions comprising trifluoroiodomethane (CF₃I) and water. The composition may consist essentially of trifluoroiodomethane (CF₃I) and water or the composition may consist of trifluoroiodomethane (CF₃I) and water.

The present inventors have found experimentally that trifluoroiodomethane (CF₃I) and water form a heterogeneous azeotropic or azeotrope-like composition.

An “azeotrope” (or “azeotropic”) composition is a unique combination of two or more components. An azeotrope can be either homogenous (which has one liquid phase) or heterogenous (which has two liquid phases). An azeotrope composition can be characterized in various ways. For example, at a given pressure, an azeotrope composition boils at a constant characteristic temperature which is either greater than the higher boiling point component (maximum boiling azeotrope) or less than the lower boiling point component (minimum boiling azeotrope). However, in the case of a heterogenous azeotrope the boiling point of the azeotrope will always be below the boiling point of the lower boiling point component. In the case of a heterogenous azeotrope then at this characteristic temperature the composition of each of the two liquid phases and the vapor phase will remain constant upon boiling. The azeotrope composition does not fractionate upon boiling or evaporation. Therefore, the components of the azeotrope composition cannot be separated during a phase change.

A heterogenous azeotrope consists of two liquid phases and one vapor phase, or one solid, one liquid, and one vapor phase, all in equilibrium. For a heterogenous azeotrope at a given temperature and pressure, the composition of each of the two liquid phases and the composition of the vapor phase remain constant. If a heterogenous azeotrope is formed, at a constant pressure the boiling point of the heterogenous azeotrope will be less than the lower boiling point component (a “minimum boiling azeotrope”).

An azeotrope composition is also characterized in that at the characteristic azeotrope temperature, the bubble point pressure of the liquid phase is identical to the dew point pressure of the vapor phase.

The behavior of an azeotrope composition is in contrast with that of a non-azeotrope composition in which during boiling or evaporation, the liquid composition changes to a substantial degree.

For the purposes of the present disclosure, an azeotrope composition is characterized as that composition which boils at a constant characteristic temperature, the temperature being lower (a minimum boiling azeotrope) than the boiling points of the two or more components, and thereby having the same composition in both the vapor and liquid phases.

One of ordinary skill in the art would understand however that at different pressures, both the composition and the boiling point of the azeotrope composition will vary to some extent. Therefore, depending on the temperature and/or pressure, an azeotrope composition can have a variable composition. The skilled person would therefore understand that composition ranges, rather than fixed compositions, can be used to define azeotrope compositions. In addition, an azeotrope may be defined in terms of exact weight percentages of each component of the compositions characterized by a fixed boiling point at a specified pressure.

Azeotrope or azeotrope-like compositions can be identified using a number of different methods.

For the purposes of this disclosure the azeotrope or azeotrope-like composition is identified experimentally using an ebulliometer (Walas, Phase Equilibria in Chemical Engineering, Butterworth-Heinemann, 1985, 533-544). An ebulliometer is designed to provide extremely accurate measurements of the boiling points of liquids by measuring the temperature of the vapor-liquid equilibrium.

The boiling points of each of the components alone are measured at a constant pressure. As the skilled person will appreciate, for a binary azeotrope or azeotrope-like composition, the boiling point of one of the components of the composition is initially measured. The second component of the composition is then added in varying amounts and the boiling point of each of the obtained compositions is measured using the ebulliometer at said constant pressure. In the case of a ternary azeotrope the initial composition would comprise of a binary blend and a third component is added in varying amounts. The boiling point of each of the obtained ternary compositions is measured using the ebulliometer at said constant pressure.

The measured boiling points are plotted against the composition of the tested composition, for example, for a binary azeotrope, the amount of the second component added to the composition, (expressed as either weight % or mole %). The presence of an azeotrope composition can be identified by the observation of a maximum or minimum boiling temperature which is greater or less than the boiling points of any of the components alone.

As the skilled person will appreciate, the identification of the azeotrope or azeotrope-like composition is made by the comparison of the change in the boiling point of the composition on addition of the second component to the first component, relative to the boiling point of the first component. Thus, it is not necessary that the system be calibrated to the reported boiling point of the particular components in order to measure the change in boiling point.

As previously discussed, at the maximum or minimum boiling point, the composition of the vapor phase will be identical to the composition of the liquid phases. The azeotrope-like composition is therefore that composition of components which provides a substantially constant minimum or maximum boiling point, that is a boiling point between about 18.0° C. and about 19.0° C. at a pressure of between about 58.0 psia and about 60.0 psia, at which substantially constant boiling point the composition of the vapor phase will be substantially identical to the composition of the liquid phases.

The present disclosure provides an azeotrope or azeotrope-like composition which comprises effective amounts trifluoroiodomethane (CF₃I) and water to form an azeotrope or azeotrope-like composition. As used herein, the term “effective amount” is an amount of each component which, when combined with the other component, results in the formation of an azeotrope or azeotrope-like mixture.

The present azeotrope or azeotrope-like compositions may consist essentially of combinations of amounts trifluoroiodomethane (CF₃I) and water or consist of combinations of amounts trifluoroiodomethane (CF₃I) and water.

As used herein, the term “consisting essentially of”, with respect to the components of an azeotrope or azeotrope-like composition or mixture, means the composition contains the indicated components in an azeotrope or azeotrope-like ratio, and may contain additional components provided that the additional components do not form new azeotrope or azeotrope-like systems. For example, azeotrope mixtures consisting essentially of two compounds are those that form binary azeotropes, which optionally may include one or more additional components, provided that the additional components do not render the mixture non-azeotropic and do not form an azeotrope with either or both of the compounds (e.g., do not form a ternary or higher azeotrope).

The present disclosure also provides a method of forming an azeotrope or azeotrope-like composition by mixing, combining, or blending, effective amounts of trifluoroiodomethane (CF₃I) and water. Any of a wide variety of methods known in the art for combining two or more components to form a composition can be used in the present methods. For example, trifluoroiodomethane (CF₃I) and water can be mixed, blended, or otherwise combined by hand and/or by machine, as part of a batch or continuous reaction and/or process, or via combinations of two or more such steps. The components can be provided in the required amounts, for example by weighing and then combining the amounts.

The azeotrope or azeotrope-like composition has a boiling point between about 18.0° C. and about 19.0° C. at a pressure of between about 58.0 psia and about 60.0 psia, and comprises, consists essentially of, or consists of, from about 47.7 wt. % to about 99.0 wt. % trifluoroiodomethane (CF₃I) and from about 1.0 wt. % to about 52.3 wt. % water, from about 60.4 wt. % to about 95.0 wt. % trifluoroiodomethane (CF₃I) and from about 5.0 wt. % to about 39.6 wt. % water, from about 70.2 wt. % to about 90.0 wt. % trifluoroiodomethane (CF₃I) and from about 10.0 wt. % to about 29.8 wt. % water, or the azeotrope or azeotrope-like composition may consist essentially of about 77.0 wt. % trifluoroiodomethane (CF₃I) and about 23.0 wt. % water.

The present disclosure also provides a composition comprising the azeotrope or azeotrope-like composition. For example, there is provided a composition comprising at least about 5 wt. % of the azeotrope or azeotrope-like composition, or at least about 15 wt. % of the azeotrope or azeotrope-like composition, or at least about 50 wt. % of the azeotrope or azeotrope-like composition, or at least about 70 wt. % of the azeotrope or azeotrope-like composition, or at least about 90 wt. % of the azeotrope or azeotrope-like composition.

The azeotrope or azeotrope-like composition comprising, consisting essentially of, or consisting of effective amounts of trifluoroiodomethane (CF₃I) and water disclosed herein may be used for separating impurities from trifluoroiodomethane (CF₃I). Such impurities may include trifluoromethane (HFC-23), chlorotrifluoromethane (CFC-13), hexafluoroethane (HFC-116), CF₂HI, CHF₂I, C₂F₅I, HCFC-22, and/or CH₃Cl, for example.

The preparation of azeotropic or azeotrope-like compositions comprising, consisting essentially of, or consisting of effective amounts of trifluoroiodomethane (CF₃I) and water allows separation techniques such as azeotropic distillation, phase separation, or fractionation, for example, to be used to remove impurities from trifluoroiodomethane (CF₃I).

In particular, an azeotrope or azeotrope-like composition comprising, consisting essentially of, or consisting of effective amounts of trifluoroiodomethane (CF₃I) and water may be formed from a composition including trifluoroiodomethane (CF₃I), water, and at least one impurity. For example, trifluoroiodomethane (CF₃I), water, or both, may be added to the composition to form the azeotrope or azeotrope-like composition. Following the formation of the azeotrope or azeotrope-like composition, the azeotrope or azeotrope-like composition may be separated from the other chemical compounds by a suitable method, such as by distillation, phase separation, or fractionation.

Following the separation, the composition may be altered in its characteristics such that the water may be removed from the composition and the trifluoroiodomethane (CF₃I) may be further purified. Suitable methods to purify the trifluoroiodomethane (CF₃I) may include distillation, liquid-liquid extraction, or exposure to a drying agent.

In one example, the present disclosure provides a method of separating impurities from trifluoroiodomethane (CF₃I), comprising the steps of providing a composition of crude trifluoroiodomethane (CF₃I) and water, modifying the relative amounts of trifluoroiodomethane (CF₃I) and water, and subjecting the composition to conditions effective to form an azeotrope or azeotrope-like composition consisting essentially of, or consisting of, effective amounts of trifluoroiodomethane (CF₃I) and water, and separating the azeotrope or azeotrope-like composition from the at least one impurity by a separation technique such as phase separation, distillation, or fractionation, for example. The step of modifying the relative amounts of trifluoroiodomethane (CF₃I) and water may involve adding trifluoroiodomethane (CF₃I) to the composition, adding water to the composition, or adding both trifluoroiodomethane (CF₃I) and water to the composition.

In another example, the present disclosure provides a method of separating impurities from trifluoroiodomethane (CF₃I), comprising the steps of providing a composition of crude trifluoroiodomethane (CF₃I), adding an effective amount of water to the composition, modifying the relative amounts of trifluoroiodomethane (CF₃I) and water, and subjecting the composition to conditions effective to form an azeotrope or azeotrope-like composition consisting essentially of, or consisting of, effective amounts of trifluoroiodomethane (CF₃I) and water, and separating the azeotrope or azeotrope-like composition from the at least one impurity by a separation technique such as phase separation, distillation, or fractionation, for example. The step of modifying the relative amounts of trifluoroiodomethane (CF₃I) and water may involve adding trifluoroiodomethane (CF₃I) to the composition, adding water to the composition, or adding both trifluoroiodomethane (CF₃I) and water to the composition.

Thereafter, the azeotrope or azeotrope-like composition may be subjected to further separation or purification steps to obtain purified trifluoroiodomethane (CF₃I). Following the separation, the composition may be altered in its characteristics such that the water may be removed from the composition and the trifluoroiodomethane (CF₃I) may be further purified. Suitable methods to purify the trifluoroiodomethane (CF₃I) may include distillation, liquid-liquid extraction, or exposure to a drying agent.

The following non-limiting example serves to illustrate the invention.

### EXAMPLES

### Example 1—Vapor Liquid Equilibrium (VLE) Study at 59.92 Psia

An ebulliometer including a vacuum-jacketed tube with a condenser at its upper end was further equipped with a Quartz Thermometer. The condenser was cooled by circulating glycol-water mixture set at the desired temperature. The pressure was regulated by a pressure controller set at about 59.9 psia.

In the first run, 123.55 g of trifluoromethane (CF₃I) was charged to the boiler and the equilibrium temperature was recorded at the set pressure of 59.92 psia. Then water was added incrementally via syringe pump, and the new equilibrium temperature was recorded. In the second run, 44.92 grams of water was charged to the boiler, and the equilibrium temperature was recorded at the set pressure of 59.92 psia. Then trifluoroiodomethane (CF₃I) was added incrementally via syringe pump, and the new equilibrium temperature was recorded. The results are shown in below in Table 1, below.

TABLE 1

| Wt. % CF₃I | Wt. % Water | Temperature ° C. |
|---|---|---|
| 100.0 | 0.0 | 18.82 |
| 99.0 | 1.0 | 18.76 |
| 95.0 | 5.0 | 18.68 |
| 90.0 | 10.0 | 18.69 |
| 77.0 | 23.0 | 18.68 |
| 70.2 | 29.8 | 18.70 |
| 60.4 | 39.6 | 18.72 |
| 47.7 | 52.3 | 18.77 |
| 0.0 | 100.0 | 144.70 |

### Example 2—Separation and Purification of Trifluoroiodomethane (CF₃I)

A composition including crude trifluoroiodomethane (CF₃I), at least one impurity, and water, is purified. In a first step, the relative amounts of trifluoroiodomethane (CF₃I) and water are adjusted. The relative amounts of trifluoroiodomethane (CF₃I) and water may be adjusted by adding water, adding trifluoroiodomethane (CF₃I), or both. The composition is then exposed to effective conditions such that an azeotrope or azeotrope-like mixture is formed. The azeotrope or azeotrope-like mixture may then be separated from the at least one impurity by distillation, phase separation, or fractionation. Once the azeotrope or azeotrope-like mixture is separated from the impurity, the components of the azeotrope or azeotrope-like mixture—trifluoroiodomethane (CF₃I) and water—are separated from one another to purify the trifluoroiodomethane. The separation of trifluoroiodomethane (CF₃I) and water may then be accomplished by distillation, liquid-liquid extraction, or exposure to a drying agent.

### Aspects

Aspect 1 is a composition comprising a heterogeneous azeotrope or azeotrope-like composition consisting essentially of effective amounts of trifluoroiodomethane (CF₃I) and water.

Aspect 2 is the composition of Aspect 1, wherein the azeotrope or azeotrope-like composition has a boiling point between about 18.0° C. and about 19.0° C. at a pressure of between about 58.0 psia and about 60.0 psia.

Aspect 3 is the composition of Aspect 1 or Aspect 2, wherein the azeotrope or azeotrope-like composition consists essentially of from about 47.7 wt. % to about 99.0 wt. % trifluoroiodomethane (CF₃I) and from about 1.0 wt. % to about 52.3 wt. % water.

Aspect 4 is the composition of any of Aspects 1-3, wherein the azeotrope or azeotrope-like composition consists essentially of from about 60.4 wt. % to about 95.0 wt. % trifluoroiodomethane (CF₃I) and from about 5.0 wt. % to about 39.6 wt. % water.

Aspect 5 is the composition of any of Aspects 1-4, wherein the azeotrope or azeotrope-like composition consists essentially of from about 70.2 wt. % to about 90.0 wt. % trifluoroiodomethane (CF₃I) and from about 10.0 wt. % to about 29.8 wt. % water.

Aspect 6 is the composition of any of Aspects 1-5, wherein the azeotrope or azeotrope-like composition consists essentially of about 77.0 wt. % trifluoroiodomethane (CF₃I) and about 23.0 wt. % water.

Aspect 7 is a method of forming a heterogeneous azeotrope or azeotrope-like composition comprising the step of combining trifluoroiodomethane (CF₃I) and water to form an azeotrope or azeotrope-like composition consisting essentially of effective amounts of trifluoroiodomethane (CF₃I) and water and having a boiling point between about 18.0° C. and about 19.0° C. at a pressure of between about 58.0 psia and about 60.0 psia.

Aspect 8 is the method of Aspect 7, wherein the combining step comprises combining from about 47.7 wt. % to about 99.0 wt. % trifluoroiodomethane (CF₃I) and from about 1.0 wt. % to about 52.3 wt. % water.

Aspect 9 is the method of Aspect 7 or Aspect 8, wherein the combining step comprises combining from about 60.4 wt. % to about 95.0 wt. % trifluoroiodomethane (CF₃I) and from about 5.0 wt. % to about 39.6 wt. % water.

Aspect 10 is the method of any of Aspects 7-9, wherein the combining step comprises combining from about 70.2 wt. % to about 90.0 wt. % trifluoroiodomethane (CF₃I) and from about 10.0 wt. % to about 29.8 wt. % water

Aspect 11 is the method of any of Aspects 7-10, wherein the combining step comprises combining about 77.0 wt. % trifluoroiodomethane (CF₃I) and about 23.0 wt. % water.

Aspect 12 is a method of separating impurities from a composition including trifluoroiodomethane (CF₃I), water, and at least one impurity, comprising the steps of modifying the relative amounts of trifluoroiodomethane (CF₃I) and water and subjecting the composition to conditions effective to form a heterogeneous azeotrope or azeotrope-like composition consisting essentially of, or consisting of, effective amounts of trifluoroiodomethane (CF₃I) and water; and separating the azeotrope or azeotrope-like composition from the impurity.

Aspect 13 is the method of Aspect 12, wherein the step of modifying the relative amounts of trifluoroiodomethane (CF₃I) and water comprises adding trifluoroiodomethane (CF₃I) to the composition.

Aspect 14 is the method of Aspect 12 or Aspect 13, wherein the step of modifying the relative amounts of trifluoroiodomethane (CF₃I) and water comprises adding water to the composition.

Aspect 15 is the method of any of Aspects 12-14, wherein the step of modifying the relative amounts of trifluoroiodomethane (CF₃I) and water comprises adding both trifluoroiodomethane (CF₃I) and water to the composition.

Aspect 16 is the method of any of Aspects 12-15, further comprising, after the separation step, the additional step of purifying the trifluoroiodomethane (CF₃I).

Aspect 17 is the method of any of Aspects 12-16, wherein the step of purifying the trifluoroiodomethane (CF₃I) comprises removing water from the trifluoroiodomethane (CF₃I).

Aspect 18 is the method of any of Aspects 12-17, wherein the step of purifying the trifluoroiodomethane (CF₃I) comprises distillation.

Aspect 19 is the method of any of Aspects 12-18, wherein the step of purifying the trifluoroiodomethane (CF₃I) comprises liquid-liquid extraction.

Aspect 20 is the method of any of Aspects 12-19, wherein the step of purifying the trifluoroiodomethane (CF₃I) comprises exposing the trifluoroiodomethane (CF₃I) to a drying agent.

As used herein, the phrase “within any range defined between any two of the foregoing values” literally means that any range may be selected from any two of the values listed prior to such phrase regardless of whether the values are in the lower part of the listing or in the higher part of the listing. For example, a pair of values may be selected from two lower values, two higher values, or a lower value and a higher value.

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

As used herein, the phrase “within any range defined between any two of the foregoing values” literally means that any range may be selected from any two of the values listed prior to such phrase regardless of whether the values are in the lower part of the listing or in the higher part of the listing. For example, a pair of values may be selected from two lower values, two higher values, or a lower value and a higher value.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances that fall within the scope of the appended claims.

1. A heterogeneous azeotrope or azeotrope-like composition consisting essentially of from about 47.7 wt. % to about 99.0 wt. % trifluoroiodomethane (CF₃I) and from about 1.0 wt. % to about 52.3 wt. % water and having a boiling point between about 18.0° C. and about 19.0° C. at a pressure of between about 58.0 psia and about 60.0 psia.

2. The composition of claim 1, wherein the heterogeneous azeotrope or azeotrope-like composition consists essentially of from about 60.4 wt. % to about 95.0 wt. % trifluoroiodomethane (CF₃I) and from about 5.0 wt. % to about 39.6 wt. % water and having a boiling point between about 18.0° C. and about 19.0° C. at a pressure of between about 58.0 psia and about 60.0 psia.

3. The composition of claim 1, wherein the heterogeneous azeotrope or azeotrope-like composition consists essentially of from about 70.2 wt. % to about 90.0 wt. % trifluoroiodomethane (CF₃I) and from about 10.0 wt. % to about 29.8 wt. % water and having a boiling point between about 18.0° C. and about 19.0° C. at a pressure of between about 58.0 psia and about 60.0 psia.

4. The composition of claim 1, wherein the heterogeneous azeotrope or azeotrope-like composition consists essentially of about 77.0 wt. % trifluoroiodomethane (CF₃I) and about 23.0 wt. % water and has a boiling point between about 18.0° C. and about 19.0° C. at a pressure of between about 58.0 psia and about 60.0 psia.

5. A method of forming a heterogeneous azeotrope or azeotrope-like composition comprising the step of combining from about 47.7 wt. % to about 99.0 wt. % trifluoroiodomethane (CF₃I) and from about 1.0 wt. % to about 52.3 wt. % water to form an azeotrope or azeotrope-like composition consisting essentially of trifluoroiodomethane (CF₃I) and water and having a boiling point between about 18.0° C. and about 19.0° C. at a pressure of between about 58.0 psia and about 60.0 psia.

6. The method of claim 5, wherein the combining step comprises combining from about 60.4 wt. % to about 95.0 wt. % trifluoroiodomethane (CF₃I) and from about 5.0 wt. % to about 39.6 wt. % water and having a boiling point between about 18.0° C. and about 19.0° C. at a pressure of between about 58.0 psia and about 60.0 psia.

7. The method of claim 5, wherein the combining step comprises combining from about 70.2 wt. % to about 90.0 wt. % trifluoroiodomethane (CF₃I) and from about 10.0 wt. % to about 29.8 wt. % water and having a boiling point between about 18.0° C. and about 19.0° C. at a pressure of between about 58.0 psia and about 60.0 psia.

8. The method of claim 5, wherein the combining step comprises combining about 77.0 wt. % trifluoroiodomethane (CF₃I) and about 23.0 wt. % water and having a boiling point between about 18.0° C. and about 19.0° C. at a pressure of between about 58.0 psia and about 60.0 psia.

9. A method of separating at least one impurity from a composition including trifluoroiodomethane (CF₃I), water, and at least one impurity, comprising the steps of:
forming a heterogeneous azeotrope or azeotrope-like composition consisting essentially of from about 47.7 wt. % to about 99.0 wt. % trifluoroiodomethane (CF₃I) and from about 1.0 wt. % to about 52.3 wt. % water and having a boiling point between about 18.0° C. and about 19.0° C. at a pressure of between about 58.0 psia and about 60.0 psia; and
separating the heterogeneous azeotrope or azeotrope-like composition from the at least one impurity.

10-12. (canceled)

13. The method of claim 9, further comprising, after the separating step, a step of purifying the trifluoroiodomethane (CF₃I).

14. The method of claim 13, wherein the step of purifying the trifluoroiodomethane (CF₃I) comprises removing water from the trifluoroiodomethane (CF₃I).

15. The method of claim 13, wherein the step of purifying the trifluoroiodomethane (CF₃I) comprises distillation.

16. The method of claim 13, wherein the step of purifying the trifluoroiodomethane (CF₃I) comprises liquid-liquid extraction.

17. The method of claim 13, wherein the step of purifying the trifluoroiodomethane (CF₃I) comprises exposing the trifluoroiodomethane (CF₃I) to a drying agent.
